(19) United States
(12) Patent Application Publication
(10) Pub. No.: US 2010/0057141 A1
(43) Pub. Date: Mar. 4, 2010

(54) MULTI-ANCHOR ANTI-BACK OUT MECHANISM AND METHOD

(75) Inventors: Mahmoud F. Abdelgany, Rockaway, NJ (US); YoungHoon Oh, Montville, NJ (US)

Correspondence Address: Rahman LLC, 10025 Governor Warfield Parkway, Suite 110, Columbia, MD, 21044, US

(73) Assignee: Custom Spine, Inc., Parsippany, NJ (US)

(21) Appl. No.: 12/199,118

(22) Filed: Aug. 27, 2008

(51) Int. Cl. A61B 17/04

(52) U.S. Cl. 606/310; 606/301

(57) ABSTRACT

A medical device and apparatus comprising a primary anchor component comprising a circular end comprising a first opening; a primary shaft extending from the circular end and comprising a second opening; a primary tip end positioned opposite to the circular end; and an angled passageway connecting the first opening to the second opening. The angled passageway comprises means for engaging the notch causing the secondary shaft to bend at the notch. The apparatus further comprises a secondary anchor component insertable through the primary anchor component, wherein the secondary anchor component comprises a threaded end; a secondary shaft extending from the threaded end and comprising a notch that permits the secondary shaft to bend; and a secondary tip end positioned below the notch. The secondary tip end extends out of the second opening when the secondary anchor component is inserted into the primary anchor component.

### BACKGROUND

1. Technical Field

The embodiments herein generally relate to medical devices, and, more particularly, to spinal implant mechanisms.

2. Description of the Related Art

The bone-screw interfaces of conventional spinal implants are typically comprised of threaded mechanisms or hook mechanisms. However, the conventional implants generally cannot adequately resist the outside compound forces, which are containing rotational and/or axial forces.

### SUMMARY

In view of the foregoing, an embodiment herein provides an apparatus comprising a primary anchor component comprising a circular end comprising a first opening; a primary shaft extending from the circular end and comprising a second opening; a primary tip end positioned opposite to the circular end; and an angled passageway connecting the first opening to the second opening. Preferably, the circular end comprises a bulbous socket. Furthermore, the angled passageway preferably comprises means for engaging the notch causing the secondary shaft to bend at the notch. The apparatus further comprises a secondary anchor component insertable through the primary anchor component, wherein the secondary anchor component comprises a threaded end; a secondary shaft extending from the threaded end and comprising a notch that permits the secondary shaft to bend; and a secondary tip end positioned below the notch. The secondary tip end may extend out of the second opening when the secondary anchor component is inserted into the primary anchor component.

The secondary anchor component may comprise nitinole. Additionally, the primary shaft may comprise a third opening. Moreover, the secondary shaft may comprise a bifurcated secondary tip end. Furthermore, in this embodiment the bifurcated secondary tip end preferably extends out of the second opening and the third opening when the secondary anchor component is inserted into the primary anchor component. Also, the secondary shaft may comprise threads. Moreover, the primary anchor component may comprise any of a bone screw, a nail, and a curved nail.

Another embodiment provides a medical device comprising a first anchor and a second anchor each insertable into a bone, wherein the first anchor bends when inserted into the second anchor. Preferably, the first anchor comprises a threaded end; a cylindrical portion extending from the threaded end and comprising a notch that permits the shaft to bend; and a lower end positioned below the notch. Moreover, the second anchor preferably comprises a circular end comprising a first opening; a shaft extending from the circular end and comprising a second opening; a tip end positioned opposite to the circular end; and a curved passageway connecting the first opening to the second opening. Preferably, the lower end extends out of the second opening when the first anchor is inserted into the second anchor.

Additionally, the shaft may comprise a third opening. Furthermore, the cylindrical portion may comprise a bifurcated lower end, wherein the bifurcated lower end may extend out of the second opening and the third opening when the first anchor is inserted into the second anchor. Preferably, the angled passageway comprises means for engaging the notch causing the cylindrical portion of the first anchor to bend at the notch.

Another embodiment provides a method comprising inserting a first bone anchoring component into a bone, wherein the first bone anchoring component comprises a threaded end; a cylindrical portion extending from the threaded end and comprising a notch that permits the cylindrical portion to bend; and a lower end positioned below the notch; inserting the first bone anchoring component into a second bone anchoring component, wherein the second bone anchoring component comprises a circular end comprising a first opening; a shaft extending from the circular end and comprising a second opening; a tip end positioned opposite to the circular end; and an angled passageway connecting the first opening to the second opening; and bending the first bone anchoring component, wherein the first bone anchoring component bends when inserted into the second bone anchoring component. Additionally, the shaft may comprise a third opening, and wherein the cylindrical portion comprises a bifurcated lower end.

These and other aspects of the embodiments herein will be better appreciated and understood when considered in conjunction with the following description and the accompanying drawings. It should be understood, however, that the following descriptions, while indicating preferred embodiments and numerous specific details thereof, are given by way of illustration and not of limitation. Many changes and modifications may be made within the scope of the embodiments herein without departing from the spirit thereof, and the embodiments herein include all such modifications.

### BRIEF DESCRIPTION OF THE DRAWINGS

The embodiments herein will be better understood from the following detailed description with reference to the drawings, in which:

FIG. 1(A) illustrates a perspective view of an anti-back out mechanism according to a first embodiment herein;

FIG. 1(B) illustrates a cross-sectional view of the anti-back out mechanism of FIG. 1(A) according to the first embodiment herein;

FIG. 2(A) illustrates a side view of the primary anchor of the anti-back out mechanism of FIG. 1(A) according to the first embodiment herein;

FIG. 2(B) illustrates a cross-sectional view of the primary anchor of FIG. 2(A) according to the first embodiment herein;

FIG. 2(C) illustrates a top view of the primary anchor of FIG. 2(A) according to the first embodiment herein;

FIG. 3(A) illustrates a perspective view of an anti-back out mechanism according to a second embodiment herein;

FIG. 3(B) illustrates a cross-sectional view of the anti-back out mechanism of FIG. 3(A) according to the second embodiment herein;

FIG. 4(A) illustrates a side view of the primary anchor of the anti-back out mechanism of FIG. 3(A) according to the second embodiment herein;

FIG. 4(B) illustrates a cross-sectional view of the primary anchor of FIG. 4(A) according to the second embodiment herein;

FIG. 4(C) illustrates a top view of the primary anchor of FIG. 4(A) according to the second embodiment herein;

FIG. 5(A) illustrates a front view of the secondary anchor of FIG. 1(A) according to the first embodiment herein;

FIG. 5(B) illustrates a side view of the secondary anchor of FIG. 5(A) according to the first embodiment herein;

FIG. 5(C) illustrates a top view of the secondary anchor of FIG. 5(A) according to the first embodiment herein;

FIG. 6(A) illustrates a perspective view of an anti-back out mechanism according to a third embodiment herein;

FIG. 6(B) illustrates a cross-sectional view of the anti-back out mechanism of FIG. 6(A) according to the third embodiment herein;

FIG. 7(A) illustrates a front view of the primary anchor of FIG. 6(A) according to the third embodiment herein;

FIG. 7(B) illustrates a side view of the primary anchor of FIG. 7(A) according to the third embodiment herein;

FIG. 7(C) illustrates a top view of the primary anchor of FIG. 7(A) according to the third embodiment herein;

FIG. 8(A) illustrates a perspective view of an anti-back out mechanism according to a fourth embodiment herein;

FIG. 8(B) illustrates a cross-sectional view of the anti-back out mechanism of FIG. 8(A) according to the fourth embodiment herein;

FIG. 9(A) illustrates a front view of the primary anchor of FIG. 8(A) according to the fourth embodiment herein;

FIG. 9(B) illustrates a side view of the primary anchor of FIG. 9(A) according to the fourth embodiment herein;

FIG. 9(C) illustrates a top view of the primary anchor of FIG. 9(A) according to the fourth embodiment herein;

FIG. 10(A) illustrates a front view of the secondary anchor of FIGS. 6(A) and 8(A) according to the third embodiment herein;

FIG. 10(B) illustrates a side view of the secondary anchor of FIG. 10(A) according to the third embodiment herein;

FIG. 10(C) illustrates a top view of the secondary anchor of FIG. 10(A) according to the third embodiment herein; and

FIG. 11 is a flow diagram illustrating a method according to an embodiment described herein.

### DETAILED DESCRIPTION OF EMBODIMENTS

The embodiments herein and the various features and advantageous details thereof are explained more fully with reference to the non-limiting embodiments that are illustrated in the accompanying drawings and detailed in the following description. Descriptions of well-known components and processing techniques are omitted so as to not unnecessarily obscure the embodiments herein. The examples used herein are intended merely to facilitate an understanding of ways in which the embodiments herein may be practiced and to further enable those of skill in the art to practice the embodiments herein. Accordingly, the examples should not be construed as limiting the scope of the embodiments herein.

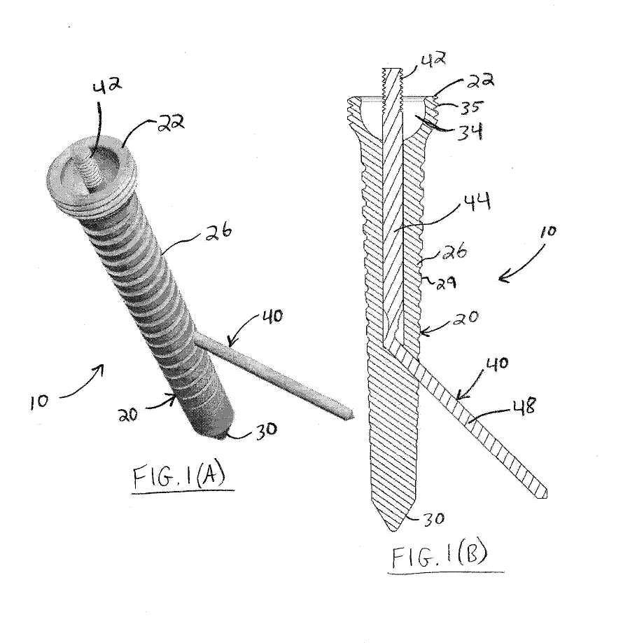

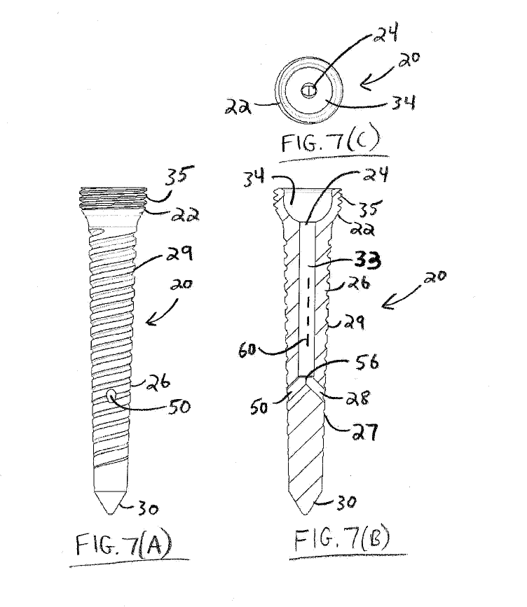

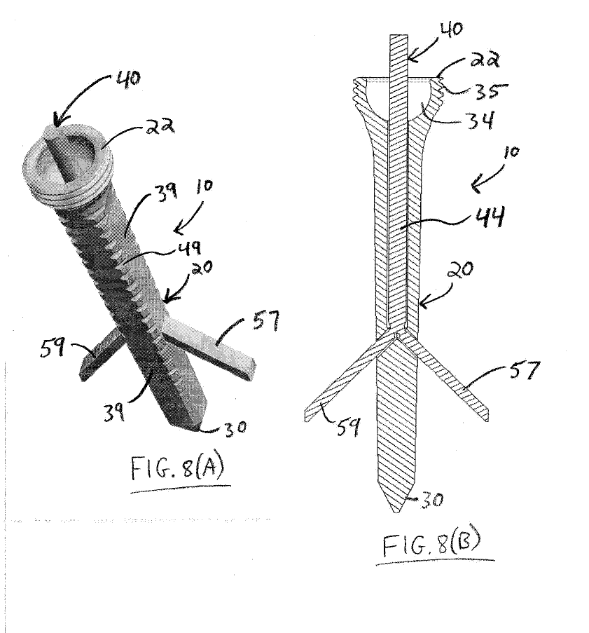

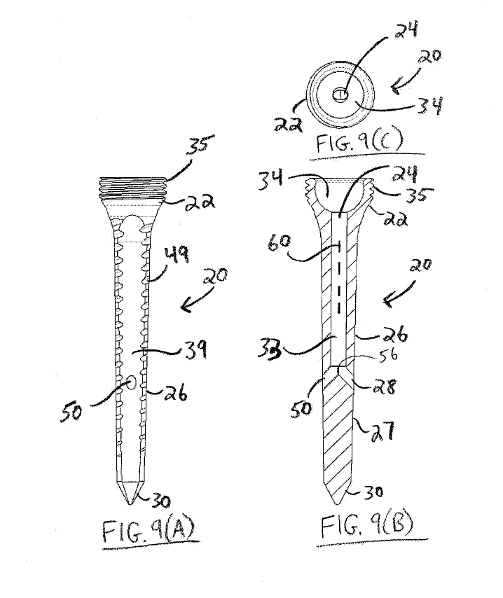

The embodiments herein provide an implant medical device configured as an anti-pull out support between bone and a bone anchor while greatly allowing a bone anchor to significantly resist all motion within bone. The device is configured to allow for multi-point anchoring using multi-anchor components. Referring now to the drawings, and more particularly to FIGS. 1(A) through 11, where similar reference characters denote corresponding features consistently throughout the figures, there are shown preferred embodiments.

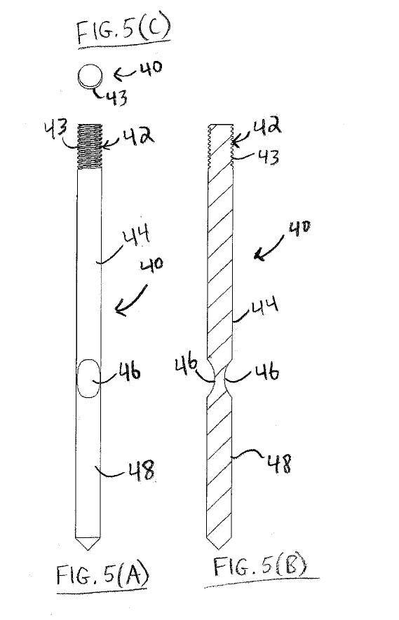

FIGS. 1(A) through 10(C) illustrate components of an apparatus 10 according to various embodiments herein, wherein the apparatus 10 comprises a hollow primary anchor component 20 comprising a circular end 22 comprising a first opening 24; a primary shaft 26 extending from the circular end 22 and comprising a second opening 28; a primary tip end 30 positioned opposite to the circular end 22; and an angled or curved passageway 32 connecting the first opening 24 to the second opening 28. As shown further illustrated in FIGS. 5(A) through 5(C), the apparatus 10 further comprises a secondary anchor component 40 insertable through the primary anchor component 20, wherein the secondary anchor component 40 comprises a threaded end 42 comprising threads 43; a secondary shaft 44 extending from the threaded end 42 and comprising a notch 46 that permits the secondary shaft 44 to bend; and a secondary tip end 48 positioned below the notch 46. The passageway 32 is dimensioned and configured to allow the secondary anchor component 40 to pass through and diverge from the main longitudinal axis 60 of the primary anchor component 20.

The circular end 22 comprises a bulbous socket 34 and has threads 35 configured around the outside of the socket 34. The angled passageway 32 comprises means 56 for engaging the notch 46 causing the secondary shaft 44 to bend at the notch 46. The means 56 may include a trigger, a point, or any mechanism that engages the secondary shaft 44 and causes it to bend at the notch 46. The secondary tip end 48 extends out of the second opening 28 when the secondary anchor component 40 is inserted into the primary anchor component 20.

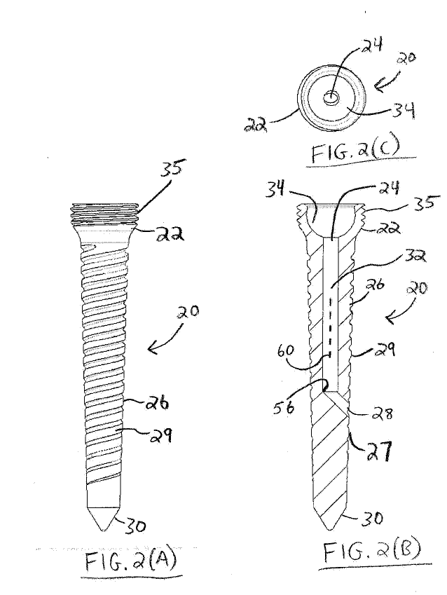

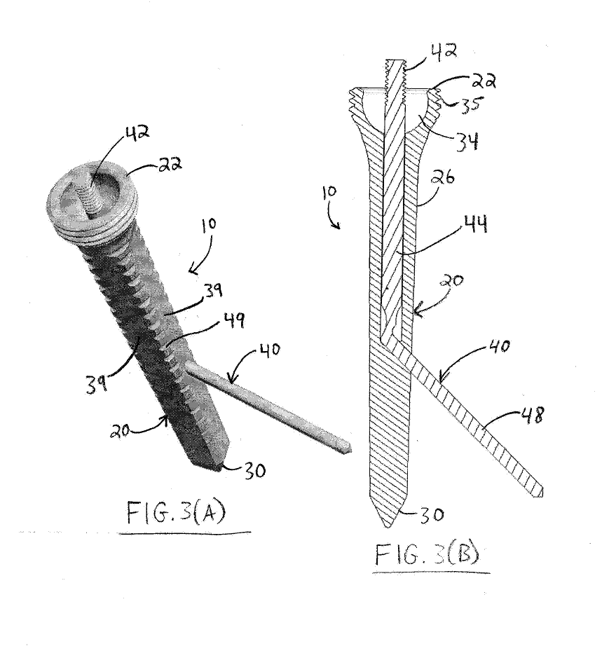

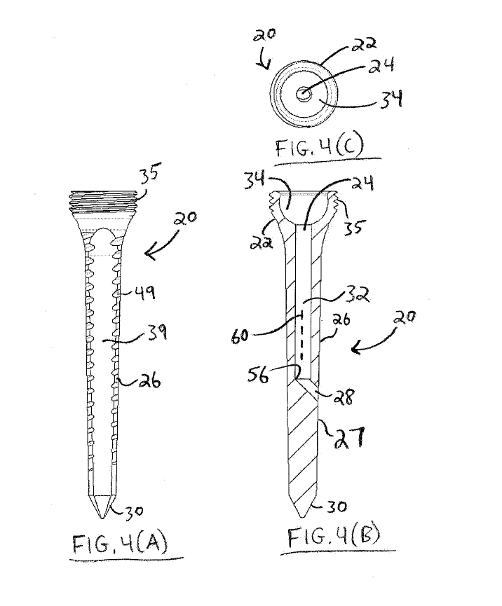

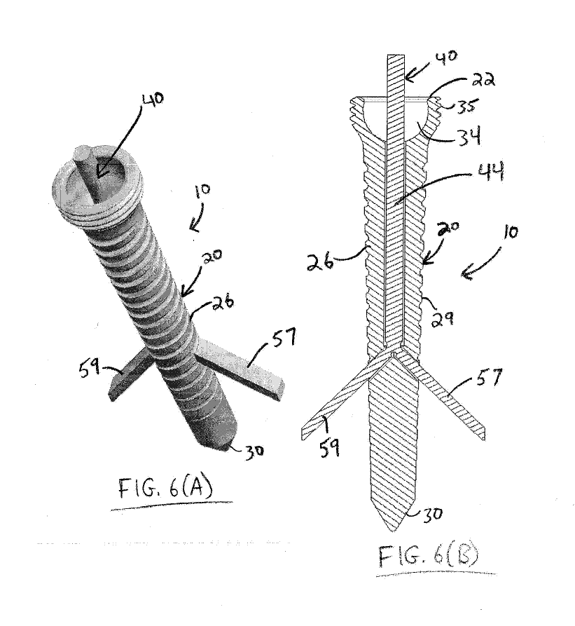

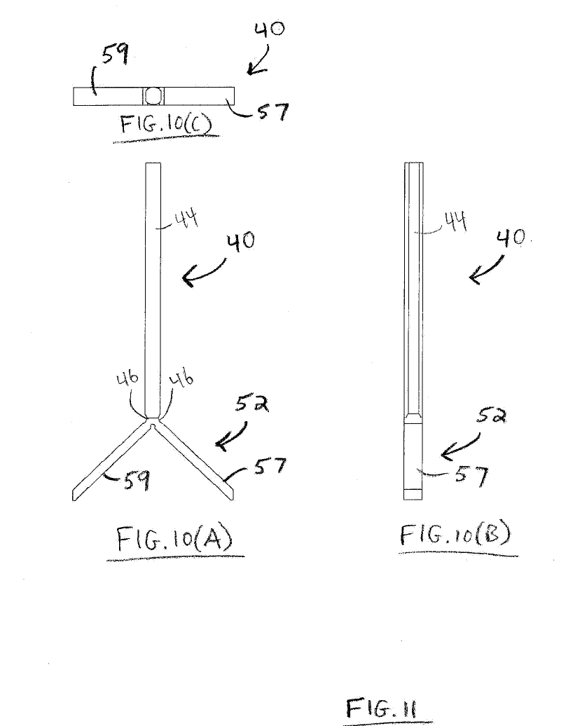

The secondary anchor component 40 may comprise nitinole (NiTi). According to the first embodiment shown in FIGS. 1(A) through 2(B), the primary shaft 26 comprises uniformly configured threads 29 and one secondary tip end 48. In a second embodiment, shown in FIGS. 3(A) through 4(C), the primary shaft 26 comprises flat outer surfaces 39 separated by threads 49. In other embodiments, shown in FIGS. 6(A) through 10(CB), the primary shaft 26 comprises a third opening 50. Here, the secondary shaft 44 comprises a bifurcated secondary tip end 52 comprising two anchors 57, 59. The bifurcated secondary tip end 52 extends out of the second opening 28 and the third opening 50 when the secondary anchor component 40 is inserted into the primary anchor component 20 and the secondary shaft 44 bends at the notch 46. In FIGS. 10(A) through 10(C) the secondary anchor component 40 is shown in its open configuration. When initially inserted into the first opening 24 of the primary anchor component 20 and into the bifurcated passageway 33, the secondary anchor component 40 is in its closed configuration, in which the two anchors 57, 59 are pushed together. Then, once the secondary tip end 52 reaches the means 56 for engaging the notch 46, the secondary tip end 52 separates the two anchors 57, 59 into a Y-shaped open configuration, and the two anchors 57, 59 exit out of the second opening 28 and third opening 50. In the bifurcated embodiment, the primary anchor component 20 comprises a bifurcated passageway 33. The secondary shaft 44 may comprise threads (not shown) towards the secondary tip end 52. The primary anchor component 20 may be configured as any of a bone screw, a nail, and a curved nail.

Both anchors 20, 40 are driven through bone (not shown). The passageway 32 in the primary anchor component 20 from the first opening 24 is initially generally parallel to the longitudinal axis 60 of the primary anchor component 20, then this passageway 32 (cannulation) diverts towards the side 25 of the primary anchor component 20. The outlet point(s) (second opening 28 and third opening 50) of passageway(s) 32, 33 is located on one or more sides of the lower portion 27 of the primary anchor component 20, which is driven into bone (not shown). The secondary anchor component 40 behaves as a supporting structure, which prevents the primary anchor component 20 from disengaging, rotating, or pivoting in bone (not shown).

During operation of the device 10, the primary anchor 20 is inserted into bone (not shown) using an appropriate insertion tool (not shown). When the primary anchor 20 seats on its intended location, then the secondary anchor 40 is inserted through the first opening 24 and through the angled or curved passageway 32, 33. At this time, the connection mechanism (i.e., thread 35) of the secondary anchor 40 is engaged to the insertion tool (not shown). As the secondary tip end 52 of the secondary anchor 40 is passing through the angled junction of the passageway 32, 33, the secondary anchor 40 is articulated to follow the path of the angled (curved) passageway 32, 33. This articulation is possible with the notch 46 on the secondary anchor 40. When the secondary anchor 40 is placed at the desired position, the connection between the insertion tool (not shown) and the secondary anchor 40 is disengaged.

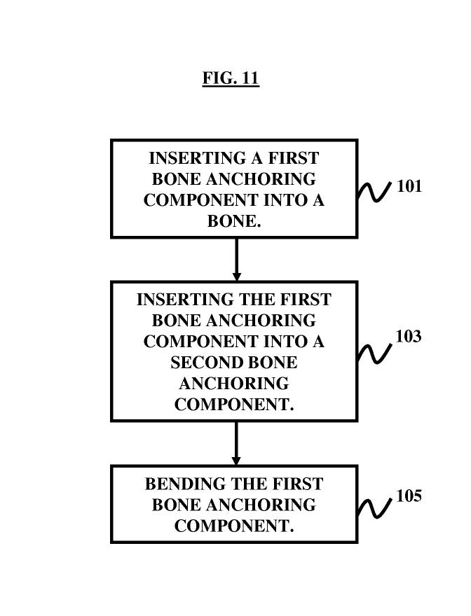

FIG. 11, with reference to FIGS. 1(A) through 10(B) is a flow diagram illustrating a method according to an embodiment herein. The method comprises inserting (101) a first bone anchoring component 40 into a bone (not shown), wherein the first bone anchoring component 40 comprises a threaded end 42; a cylindrical portion 44 extending from the threaded end 42 and comprising a notch 46 that permits the cylindrical portion 44 to bend; and a lower end 48 positioned below the notch 46; inserting (103) the first bone anchoring component into a second bone anchoring component 20, wherein the second bone anchoring component 20 comprises a circular end 22 comprising a first opening 24; a shaft 26 extending from the circular end 22 and comprising a second opening; a tip end positioned opposite to the circular end; and an angled passageway connecting the first opening to the second opening 28; and bending (105) the first bone anchoring component 40, wherein the first bone anchoring component 40 bends when inserted into the second bone anchoring component 20. Additionally, the shaft 26 may comprise a third opening 50, and wherein the cylindrical portion 44 comprises a bifurcated lower end 52.

Generally, the embodiments herein contain a second 48 or third bone anchor(s) 57, 59 protruding outside of the primary bone anchor component 20 in diverging direction(s) and provides increased resistance to prevent the instrumentation from pulling out, rotating, or pivoting. Moreover, revision or removal of the implant 10 is easily revisable without compromising the main hole (not shown) in which the primary anchor component 20 is driven. The embodiments herein may be used in any surgery that requires superior fixation in bone. Furthermore, the embodiments herein may be utilized in non-fusion environments where conventional bone anchor implants often loosen within the bone.

The foregoing description of the specific embodiments will so fully reveal the general nature of the embodiments herein that others can, by applying current knowledge, readily modify and/or adapt for various applications such specific embodiments without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. Therefore, while the embodiments herein have been described in terms of preferred embodiments, those skilled in the art will recognize that the embodiments herein can be practiced with modification within the spirit and scope of the appended claims.

What is claimed is:

1. An apparatus comprising:
a primary anchor component comprising:
a circular end comprising a first opening;
a primary shaft extending from said circular end and comprising a second opening;
a primary tip end positioned opposite to said circular end; and
an angled passageway connecting said first opening to said second opening;
a secondary anchor component insertable through said primary anchor component, wherein said secondary anchor component comprises:
a threaded end;
a secondary shaft extending from said threaded end and comprising a notch that permits said secondary shaft to bend; and
a secondary tip end positioned below said notch.

2. The apparatus of claim 1, wherein said secondary anchor component comprises nitinole.

3. The apparatus of claim 1, wherein said primary shaft comprises a third opening.

4. The apparatus of claim 3, wherein said secondary shaft comprises a bifurcated secondary tip end.

5. The apparatus of claim 4, wherein said bifurcated secondary tip end extends out of said second opening and said third opening when said secondary anchor component is inserted into said primary anchor component.

6. The apparatus of claim 1, wherein said secondary shaft comprises threads.

7. The apparatus of claim 1, wherein said circular end comprises a bulbous socket.

8. The apparatus of claim 1, wherein said angled passageway comprises means for engaging said notch causing said secondary shaft to bend at said notch.

9. The apparatus of claim 1, wherein said secondary tip end extends out of said second opening when said secondary anchor component is inserted into said primary anchor component.

10. The apparatus of claim 1, wherein said primary anchor component comprises any of a bone screw, a nail, and a curved nail.

11. A medical device comprising a first anchor and a second anchor each insertable into a bone, wherein said first anchor bends when inserted into said second anchor.

12. The medical device of claim 11, wherein said first anchor comprises:
a threaded end;
a cylindrical portion extending from said threaded end and comprising a notch that permits said shaft to bend; and
a lower end positioned below said notch.

13. The medical device of claim 12, wherein said second anchor comprises:
a circular end comprising a first opening;
a shaft extending from said circular end and comprising a second opening;
a tip end positioned opposite to said circular end; and
a curved passageway connecting said first opening to said second opening.

14. The medical device of claim 13, wherein said shaft comprises a third opening.

15. The medical device of claim 14, wherein said cylindrical portion comprises a bifurcated lower end.

16. The medical device of claim 15, wherein said bifurcated lower end extends out of said second opening and said third opening when said first anchor is inserted into said second anchor.

17. The medical device of claim 13, wherein said angled passageway comprises means for engaging said notch causing said cylindrical portion of said first anchor to bend at said notch.

18. The medical device of claim 11, wherein said lower end extends out of said second opening when said first anchor is inserted into said second anchor.

19. A method comprising:
inserting a first bone anchoring component into a bone, wherein said first bone anchoring component comprises a threaded end; a cylindrical portion extending from said threaded end and comprising a notch that permits said cylindrical portion to bend; and a lower end positioned below said notch;
inserting said first bone anchoring component into a second bone anchoring component, wherein said second bone anchoring component comprises a circular end comprising a first opening; a shaft extending from said circular end and comprising a second opening; a tip end positioned opposite to said circular end; and an angled passageway connecting said first opening to said second opening; and
bending said first bone anchoring component, wherein said first bone anchoring component bends when inserted into said second bone anchoring component.

20. The method of claim 19, wherein said shaft comprises a third opening, and wherein said cylindrical portion comprises a bifurcated lower end.
